(19) United States
(12) Patent Application Publication
(10) Pub. No.: US 2017/0347944 A1
(43) Pub. Date: Dec. 7, 2017

(54) RADIO WAVE TRANSDUCER AND A METHOD FOR DETECTION OF SPINAL SEGMENTAL DYSFUNCTION

(71) Applicant: Canadian Chiropractic Centre Limited, Hong Kong (HK)

(72) Inventor: Hung Peter HONG, Hong Kong (HK)

(73) Assignee: Canadian Chiropractic Centre Limited

(21) Appl. No.: 15/409,537

(22) Filed: Jan. 19, 2017

(30) Foreign Application Priority Data
Jun. 2, 2016 (HK) 16106297.9

(51) Int. Cl. A61B 5/00; A61B 5/11

(52) U.S. Cl. CPC A61B 5/4566; A61B 5/1104; A61B 5/7232

(57) ABSTRACT

The invention discloses a radio wave transducer for the detection of spinal segmental dysfunction, comprising two radio wave emitters and at least one switch; the two radio wave emitters are electrically connected to the switch; each of the two radio wave emitters has a RLC circuit consisting of a resistor, an inductor and a capacitor, one end of the resistor is electrically connected to one end of the inductance, the other end of the inductance is electrically connected to one end of the capacitor, the other end of the resistor is electrically connected to one end of the switch, the other end of the capacitor is electrically connected to the other end of the switch. This invention also discloses a method for detection of spinal segmental dysfunction.

### CROSS-REFERENCE TO RELATED APPLICATIONS

This application claims priority to Hong Kong Patent Application No. 16106297.9 with a filing date of Jun. 2, 2016. The content of the aforementioned application, including any intervening amendments thereto, are incorporated herein by reference.

### TECHNICAL FIELD

The invention relates to a medical equipment, in particular relates to a radio wave transducer and a method for detection of spinal segmental dysfunction.

### BACKGROUND OF THE PRESENT INVENTION

A motor action results from a set of energetic processes leading a nerve signal via the spinal cord to effector organs. A partial or total hurt of the spinal cord leads to a degeneration of the nerve pathways that can no longer transmit signals. A spinal hurt detector is a device for detection of spinal cord. Spinal hurt detector is widely used in medical industry. According to the World Federation of Chiropractic, the number of duly qualified chiropractors worldwide in 2015 is approximately 87,900. The majority of them (73,300 approx.) are located in the US and Canada. Traditional spinal hurt detector always detects based on somatosensory evoked potential (SEP). However, SEP signal is always very weak and unreliability. The accurate identification of the segmental dysfunction is of great significance, as the correction of which is the most effective and efficient way to improve the function of the spine. Many other different types of methods, including X-ray, palpation, range of motion, muscle strength, reflexes, thermography, and surface electromyography has been used to locate this key segmental dysfunction, there is no simple, effective, efficient, day-to-day examination procedure at this time. Therefore, a spinal hurt detector with stable signal is needed.

### SUMMARY OF PRESENT INVENTION

This invention overcomes the shortages stated above by providing a radio wave transducer for the detection of spinal segmental dysfunction.

The radio wave transducer for the detection of spinal segmental comprising: two radio wave emitters and at least one switch; the two radio wave emitters are electrically connected to the switch; each of the two radio wave emitters has a RLC circuit consisting of a resistor, an inductor and a capacitor, one end of the resistor is electrically connected to one end of the inductance, the other end of the inductance is electrically connected to one end of the capacitor, the other end of the resistor is electrically connected to one end of the switch, the other end of the capacitor is electrically connected to the other end of the switch.

Furthermore, the switch alternatively turns on one of the two radio wave emitters.

Furthermore, the switch is a single-pole, double-throw (SPDT) switch.

Furthermore, the switch comprises a first end connected with a pole and two second ends, the other end of the resistor is electrically connected to the first end of the SPDT switch, the other end of the capacitor is electrically connected to the second end of the SPDT switch S.

Furthermore, the RLC circuit emits RF wave of 1-10 MHz to stimulate the cutaneous receptors.

Furthermore, each radio wave emitter further comprises an indicator light to indicate the state of the radio wave emitter.

Furthermore, there are a series of radio wave transducers for testing a nest pair of dermatomes and dermatomes further down.

Furthermore, the radio wave transducers are jointed by a relay system to selectively test an individual segment of the spine.

The Invention also provides a method for detecting spinal segmental dysfunction by using the radio wave transducer of claim 1, comprising following steps:

placing the radio wave transducer at the midline of a segment of the spine with one radio wave emitter lying on one side of the spine and the other radio wave emitter E lying on the other side of the spine, the radio wave transducer can be placed above the skin with or without actual physical contact;

turning on one radio wave emitter to send out radio wave and keeping the other radio wave emitter dormant;

stimulating one of paired spinal nerve and exciting a first tonic postural response of the body;

turning on the other radio wave emitter to send out radio wave and keeping the one radio wave emitter dormant;

stimulating the other one of the paired spinal nerve and exciting a second tonic postural response of the body;

comparing the first tonic postural response of the body and the second tonic postural response of the body.

### DESCRIPTION OF THE DRAWINGS

FIG. 1 is a circuit diagram of a radio wave transducer of one embodiment in the invention;

FIG. 2 is a circuit diagram of a series of radio wave transducer of another embodiment in the invention;

wherein: R: resistor; E: emitter; L: inductor; C: capacitor; S: switch.

### DETAILED DESCRIPTION OF PREFERRED EMBODIMENTS

To illustrate the structure and advantages of the present invention, below is the detailed description of the present invention in combination with the figures and embodiments.

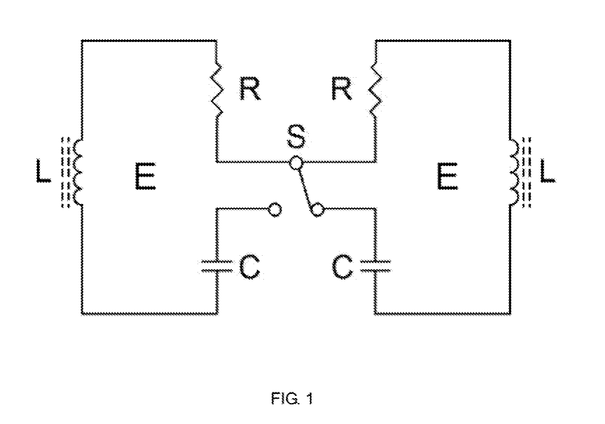

Referring to FIG. 1, a radio wave transducer for the detection of spinal segmental dysfunction, comprises two radio wave emitters E and at least one switch S. Each of the two radio wave emitters E is located on each side of the spinal. The two radio wave emitters E are electrically connected to the switch S, the switch S can alternatively turn on one of the two radio wave emitters E.

Each of the two radio wave emitters E has a RLC circuit, which consists of a passive resonant RLC resistor R, an inductor L and a capacitor C. One end of the resistor R is electrically connected to one end of the inductance L, the other end of the inductance L is electrically connected to one end of the capacitor C. The other end of the resistor R is electrically connected to one end of the switch S, the other end of the capacitor C is electrically connected to the other end of the switch S.

In another embodiment of the invention, the switch S is a single-pole, double-throw (SPDT) switch. The SPDT switch S comprises a first end connected with a pole and two second ends. The other end of the resistor R is electrically connected to the first end of the SPOT switch S, the other end of the capacitor C is electrically connected to the second end of the SPOT switch S. When the radio wave transducer is placed at the midline of a segment of the spine, one radio wave emitter E lying on one side of the spine sends out radio wave to stimulate the skin underneath, and the one that rests on the other side is dormant.

The radio wave transducer selectively stimulates one of the paired spinal nerve. This unilateral radio wave stimulation excites the tonic postural response of the body. The RLC circuit emits RF wave of 1-10 MHz to stimulate the cutaneous receptors. Preferably, the RLC circuit emits RF wave of 5 MHz.

Spinal segmental dysfunction can be identified among the normal segments by comparing how bilateral tonic postural muscles respond under the influence of device.

In one embodiment of the invention, each radio wave emitter E further comprises an indicator light to indicate the state of the radio wave emitter E.

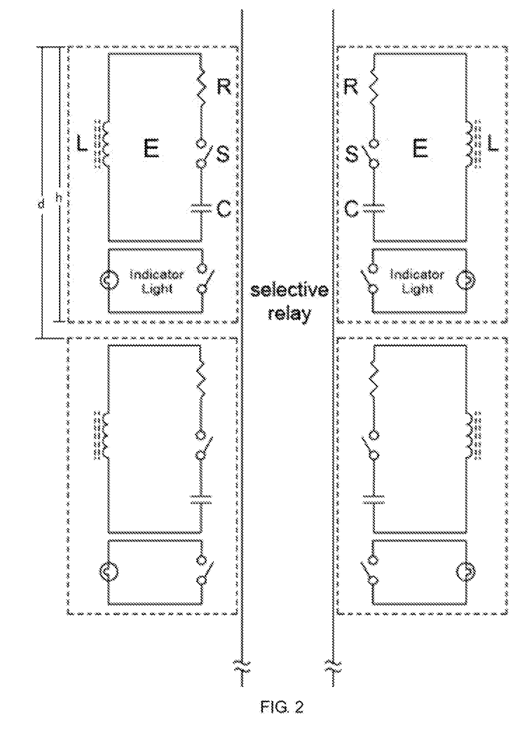

Referring to FIG. 2, in another embodiment of the invention, there are a series of radio wave transducers for testing the next pair of dermatomes and dermatomes further down.

These radio wave transducers can be jointed by a relay system to selectively testing individual segment of the spine.

In one embodiment of the invention, the transducers can be placed in an overlapped manner for the ease of detecting the spinal segmental dysfunction. As shown in FIG. 2, h is the height of the transducer, d is the distance of one below the other. The distance d can be greater or smaller than the height of an individual transducer.

This invention also provides a method for detecting spinal segmental dysfunction, which comprises the steps:

placing the radio wave transducer at the midline of a segment of the spine with one radio wave emitter E lying on one side of the spine and the other radio wave emitter E lying on the other side of the spine, the radio wave transducer can be placed above the skin with or without actual physical contact;

turning on one radio wave emitter E to send out radio wave and keeping the other radio wave emitter E dormant;

stimulating one of paired spinal nerve and exciting a first tonic postural response of the body:

turning on the other radio wave emitter E to send out radio wave and keeping the one radio wave emitter E dormant;

stimulating the other one of the paired spinal nerve and exciting a second tonic postural response of the body;

comparing the first tonic postural response of the body and the second tonic postural response of the body.

The foregoing examples are preferred embodiments of the present invention only and not intended to limit the present disclosure. It should be understood that, to the person skilled in the art, various modifications and improvements can be made without departing from the spirit and principle of the present disclosure, which should all be included within the scope of the present disclosure. Therefore, the protection scope of the present disclosure shall be defined by the appended claims.

I claim:

1. A radio wave transducer for the detection of spinal segmental comprising:
two radio wave emitters and at least one switch;
wherein the two radio wave emitters are electrically connected to the switch;
each of the two radio wave emitters has a RLC circuit consisting of a resistor, an inductor and a capacitor, one end of the resistor is electrically connected to one end of the inductance, the other end of the inductance is electrically connected to one end of the capacitor, the other end of the resistor is electrically connected to one end of the switch, the other end of the capacitor is electrically connected to the other end of the switch.

2. The radio wave transducer of claim 1, wherein the switch alternatively turns on one of the two radio wave emitters.

3. The radio wave transducer of claim 2, wherein the switch is a single-pole, double-throw (SPDT) switch.

4. The radio wave transducer of claim 3, wherein the switch comprises a first end connected with a pole and two second ends, the other end of the resistor is electrically connected to the first end of the SPDT switch, the other end of the capacitor is electrically connected to the second end of the SPDT switch S.

5. The radio wave transducer of claim 3, wherein the RLC circuit emits RF wave of 1-10 MHz to stimulate the cutaneous receptors.

6. The radio wave transducer of claim 1, wherein each radio wave emitter further comprises an indicator light to indicate the state of the radio wave emitter.

7. The radio wave transducer of claim 1, wherein there are a series of radio wave transducers for testing a next pair of dermatomes and dermatomes further down.

8. The radio wave transducer of claim 7, wherein the radio wave transducers are jointed by a relay system to selectively test an individual segment of the spine.

9. The radio wave transducer of claim 7, wherein the radio wave transducers are placed in an overlapped manner.

10. A method for detecting spinal segmental dysfunction by using the radio wave transducer of claim 1, comprising following steps:
placing the radio wave transducer at the midline of a segment of the spine with one radio wave emitter lying on one side of the spine and the other radio wave emitter E lying on the other side of the spine, the radio wave transducer can be placed above the skin with or without actual physical contact;
turning on one radio wave emitter to send out radio wave and keeping the other radio wave emitter dormant;
stimulating one of paired spinal nerve and exciting a first tonic postural response of the body;
turning on the other radio wave emitter to send out radio wave and keeping the one radio wave emitter dormant;
stimulating the other one of the paired spinal nerve and exciting a second tonic postural response of the body;
comparing the first tonic postural response of the body and the second tonic postural response of the body.

11. The radio wave transducer of claim 10, wherein the switch alternatively turns on one of the two radio wave emitters.

12. The radio wave transducer of claim 11 wherein the switch is a single-pole, double-throw (SPDT) switch.

13. The radio wave transducer of claim 12, wherein the switch comprises a first end connected with a pole and two second ends, the other end of the resistor is electrically connected to the first end of the SPDT switch, the other end of the capacitor is electrically connected to the second end of the SPDT switch S.

14. The radio wave transducer of claim 13, wherein the RLC circuit emits RF wave of 1-10 MHz to stimulate the cutaneous receptors.

15. The radio wave transducer of claim 10, wherein each radio wave emitter further comprises an indicator light to indicate the state of the radio wave emitter.

16. The radio wave transducer of claim 10, wherein there are a series of radio wave transducers for testing a nest pair of dermatomes and dermatomes further down.

17. The radio wave transducer of claim 16, wherein the radio wave transducers are jointed by a relay system to selectively test an individual segment of the spine.
